(19) United States
(12) Patent Application Publication
(10) Pub. No.: US 2024/0197393 A1
(43) Pub. Date: Jun. 20, 2024

(54) CATHETER SHAPE DETECTION FOR MAP AND ABLATE CATHETERS

(71) Applicant: Boston Scientific Scimed, Inc., Maple Grove, MN (US)

(72) Inventors: Amanda Christine Kingston, Waltham, MA (US); Brendan Early Koop, Ham Lake, MN (US); Nathan Paul Hagstrom, Minneapolis, MN (US); Eric Daniel Buck, Shoreview, MN (US); Matthew Ferrante, Cambridge, MA (US)

(21) Appl. No.: 18/510,160

(22) Filed: Nov. 15, 2023

Related U.S. Application Data
(60) Provisional application No. 63/387,376, filed on Dec. 14, 2022.

(51) Int. Cl. A61B 18/14; A61B 5/367

(52) U.S. Cl. CPC A61B 18/1492; A61B 5/367; A61B 2018/00577

(57) ABSTRACT

A system for facilitating an ablation in a patient's heart is disclosed. The system includes a catheter and a controller. The catheter includes an electrode assembly having spaced-apart electrodes disposed on splines to generate an electric field in the heart, the splines configurable in an expanded position wherein the plurality of spaced-apart electrodes are in a selected spatial relationship, and wherein the splines are deformable relative to the expanded configuration when subjected to a force. The controller is configured to measure an electrical signal received from an electrode of the spaced-apart electrodes in response to the electric field, the electrical signal indicative of a parameter of the electric field, and the controller configured to determine a deformation of the splines relative to the expanded position based on the measured electrical signal.

### CROSS-REFERENCE TO RELATED APPLICATIONS

This application claims the benefit of U.S. Provisional Application No. 63/387,376, entitled “CATHETER SHAPE DETECTION FOR MAP AND ABLATE CATHETERS,” and filed Dec. 14, 2022, which is hereby incorporated by reference in its entirety.

### TECHNICAL FIELD

The present disclosure relates to medical systems and methods for facilitating ablation of tissue in a patient. More specifically, the present disclosure relates to medical systems and methods for facilitating ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques can indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells.

### SUMMARY

In Example 1, a system for facilitating an ablation in a patient's heart, the system comprising a catheter and a controller. The catheter includes an electrode assembly having a plurality of spaced-apart electrodes disposed on a plurality of splines to generate an electric field in the heart, the splines configurable in an expanded position wherein the plurality of spaced-apart electrodes are in a selected spatial relationship, and wherein the splines are deformable relative to the expanded configuration when subjected to a force. The controller is configured to measure an electrical signal received from an electrode of the plurality of spaced-apart electrodes in response to the electric field, the electrical signal indicative of a parameter of the electric field, and the controller configured to determine a deformation of the splines relative to the expanded position based on the measured electrical signal.

In Example 2, the system of Example 1, wherein the deformation of the splines is determined based on a determination, from the measured electrical signal, of a location of the plurality of electrodes.

In Example 3, the system of any of Examples 1-2, wherein the deformation of the splines is determined from a distance between the plurality of spaced-apart electrodes based on the measured electrical signal.

In Example 4, the system of any of Examples 1-3, wherein the controller is configured to measure a plurality of electrical signals received from the plurality of spaced-apart electrodes in response to the electric field, the plurality of electrical signals indicative of parameters of the electric field.

In Example 5, the system of any of Examples 1-3, wherein the determination of the deformation includes a determination of an amount of deformation of the splines relative to the expanded position.

In Example 6, the system of Example 5, wherein the controller is configured to generate a visualization of a gradient based on the amount of deformation of the splines relative to the expanded position.

In Example 7, the system of any of Examples 5-6, wherein the controller is configured to generate a visualization of the deformation of the splines.

In Example 8, the system of Example 7, wherein the controller is configured to highlight a deformed spline of the plurality of splines in the visualization of the deformation of the splines.

In Example 9, the system of any of Examples 7-8, wherein the controller is configured to generate the visualization of the deformation of the splines on an electroanatomical map of a heart.

In Example 10, the system of any of Examples 1-9, wherein the controller is configured to determine the deformation of the splines relative to the expanded position in substantially real time.

In Example 11, the system of any of Examples 1-10, wherein the determination of the deformation includes a determination of distension of tissue.

In Example 12, the system of any of Examples 1-11, wherein the catheter includes a shaft having a distal region, and the plurality of splines form a basket in the expanded position, wherein each of the plurality of splines includes a proximal end and a distal end, the basket coupled to the distal region wherein the distal ends of the splines form a distal tip region of the basket.

In Example 13, the system of Example 12, wherein the plurality of electrodes includes a measurement electrode disposed within the basket and configured to not contact tissue when the catheter is in the expanded position.

In Example 14, the system of Example 13, wherein the plurality of electrodes includes a distal indifferent electrode disposed within the basket and coupled to the distal tip region.

In Example 15, the system of any of Examples 12-14, wherein the plurality of electrodes includes a shaft electrode disposed on the distal region of the shaft and proximal to the basket.

In Example 16, a system for facilitating an ablation, the system comprising a catheter and a controller. The catheter including an electrode assembly having a plurality of spaced-apart electrodes disposed on a plurality of splines to generate an electric field, the splines configurable in an expanded position wherein the plurality of spaced-apart electrodes are in a selected spatial relationship, and wherein the splines are deformable relative to the expanded position when subjected to a force. The controller configured to measure an electrical signal received from an electrode of the plurality of spaced-apart electrodes in response to the electric field, the electrical signal indicative of a parameter of the electric field, and the controller configured to determine a deformation of the splines relative to the expanded position based on the measured electrical signal.

In Example 17, the system of Example 17, wherein the catheter includes a shaft having a distal region, and the plurality of splines form a basket in the expanded position, wherein each of the plurality of splines includes a proximal end and a distal end, the basket coupled to the distal region wherein the distal ends of the splines form a distal tip region of the basket.

In Example 18, the system of Example 17, wherein the plurality of electrodes includes a measurement electrode disposed within the basket and configured to not contact tissue when the catheter is in the expanded position.

In Example 19, the system of Example 18, wherein the measurement electrode provides the electrical signal.

In Example 20, the system of Example 18, wherein the plurality of electrodes includes a distal indifferent electrode disposed within the basket and coupled to the distal tip region, and the controller is configured to determine a distance between the measurement electrode and the distal indifferent electrode.

In Example 21, the system of Example 16, wherein the deformation of the splines is determined based on a determination, from the measured electrical signal, of a location of each of the plurality of electrodes.

In Example 22, the system of Example 16, wherein the determination of the deformation includes a determination of an amount of deformation of the splines relative to the expanded position.

In Example 23, the system of Example 22, wherein the controller is configured to determine an amount of the force applied to the splines based on the determination of an amount of deformation.

In Example 24, the system of Example 16, wherein the controller is configured to generate a visualization based on the deformation.

In Example 25, the system of Example 24, wherein the controller is configured to generate a visualization of a gradient based on the amount of deformation of the splines relative to the expanded position.

In Example 26, the system of Example 24, wherein the controller is configured to highlight a deformed spline of the plurality of splines.

In Example 27, the system of claim 24, wherein the controller is configured to generate the visualization of the deformation on an electroanatomical map of a heart.

In Example 28, an electroporation catheter for use with tissue, the electroporation catheter comprising an elongated shaft having a distal region and an electrode assembly operably coupled to the distal region. The electrode assembly having a plurality of spaced-apart electrodes disposed on a plurality of splines to generate an electric field, the splines configurable in an expanded position wherein the plurality of spaced-apart electrodes are in a selected spatial relationship, and wherein the splines are deformable relative to the expanded position when subjected to a force. The plurality of splines form a basket defining a cavity in the expanded position, wherein each of the plurality of splines includes a proximal end and a distal end, the basket coupled to the distal region wherein the distal ends of the splines form a distal tip region of the basket, the shaft having a distal basket region extending into and terminating within the cavity. The plurality of electrodes includes a measurement electrode disposed within the basket on the distal basket region of the shaft, the measurement electrode configured to not contact the tissue when the splines are in the expanded position.

In Example 29, the catheter of Example 28, wherein the plurality of electrodes includes a distal indifferent electrode disposed within the basket and coupled to the distal tip region, the distal indifferent electrode spaced-apart from the shaft and the measurement electrode.

In Example 30, the catheter of Example 29, wherein the plurality of electrodes includes a shaft electrode disposed on the distal region of the shaft and proximal to the basket.

In Example 31, a process for facilitating an ablation in a patient's heart. the process includes generating an electric field with a catheter in the heart, the catheter including an electrode assembly having a plurality of spaced-apart electrodes disposed on a plurality of splines to generate the electric field, the splines configurable in an expanded position wherein the plurality of spaced-apart electrodes are in a selected spatial relationship, and wherein the splines are deformable relative to the expanded position when subjected to a force. The process includes measuring an electrical signal received from an electrode of the plurality of spaced-apart electrodes in response to the electric field, the electrical signal indicative of a parameter of the electric field. The process includes determining a deformation of the splines relative to the expanded position based on the measured electrical signal wherein the deformation of the splines is determined based on determining, from the measured electrical signal, a location of each of the plurality of electrodes with respect to the electrode assembly.

In Example 32, the process of Example 31, and further including determining an amount of the force applied to the catheter based on the determination of an amount of deformation.

In Example 33, the process of Example 31, and further including generating, on a graphical display, a visualization based on the deformation.

In Example 34, the process of Example 33, wherein generating the visualization includes generating the visualization of the deformation of the splines on an electroanatomical map of a heart.

In Example 35, the process of Example 31, wherein determining deformation further includes determining an amount of tissue distension based on a determined amount of the plurality of electrodes in contact with the heart, a determined location of the catheter within the heart, and a determined amount of deformation.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 is a schematic diagram illustrating a portion of an example catheter that can be used with the example electrophysiology system of FIG. 1.

FIG. 3A is a schematic diagram illustrating a portion of example catheter of FIG. 2 in a chamber of a patient's heart of FIG. 1, the catheter in an expanded position.

FIG. 3B is a schematic diagram illustrating the portion of the example catheter of FIG. 3A, the catheter in a first deformed state from the expanded position.

FIG. 3C is a schematic diagram illustrating the portion of the example catheter of FIG. 3A, the catheter in a second deformed state from the expanded position in which the second deformed state includes a greater amount of deformation than the first deformed state from the expanded position.

FIG. 4 is a block diagram illustrating an example controller for use with the example electrophysiology system of FIG. 1.

FIG. 5 is a flow diagram illustrating an example configuration of the example controller of FIG. 4.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

As applied to electrophysiology systems, irreversible electroporation uses high voltage, short pulses to kill cells such as myocardium through apoptosis while sparing other adjacent tissues including the esophageal vascular smooth muscle and endothelium. Irreversible electroporation treatment can be delivered in multiple therapy sections. A therapy section, which may have a duration on the order of milliseconds, may include a plurality of electrical pulses, such as a few dozen pulses, generated and delivered by an electroporation device, which is powered by an electroporation generator. In one example, the electroporation device is disposed near a tip of a catheter and is obscured from the naked eye when deployed. Determining locations for irreversible electroporation ablation during procedures can be difficult due to the issues associated with acute visualization or data indicating whether electroporation mapping or ablation mechanisms, such as electrode assemblies on distal regions of catheters, are in contact with tissues.

To determine the electrode positions or electrode assembly position of an electroporation ablation catheter in a conductive medium, such as an intracardiac space like the chamber of a patient's heart, many clinicians prefer the use of an electroanatomical mapping system over the use of fluoroscopy, which may lessen the time a patient is exposed to radiation. Electroanatomical mapping systems can be employed to create real-time, three-dimensional view of the heart and electrical activity within the heart. Some examples of electroanatomical mapping systems employ multielectrode basket catheters to rapidly record electrical activity for a multitude of positions within the heart, which can make the electroanatomical maps more accurate. Such catheters can include self-expanding splines having a plurality of electrodes. The splines are arranged in a three-dimensional shape like an inflated balloon that allows the catheter to conform to the shape and movement of the chambers of the heart.

Information regarding how or whether the catheter is in contact with the tissue is helpful for mapping and ablation procedures in electrophysiology systems. Also, information regarding distension of tissue in contact with the catheter is helpful in creating lesions and determining catheter interactions with the anatomy. Such information can be applied to determine completeness of the electroanatomical map and can provide a confidence as to the accuracy of signals being displayed in certain anatomical locations. With such information, a clinician may also be able to avoid certain structures of the heart or esophagus. Still further, information regarding whether the catheter is in contact with tissue can inform as to whether an ablation location is likely to result in an effective lesion.

Providing an indication on whether a catheter is in contact with tissue or an amount of contact with tissue, and what features of the catheter are in contact, can provide for a more effective mapping and ablation in electrophysiology procedures. Methods to determine force in catheters have met with difficulties. For example, force sensors on distal ends of catheters can use valuable space in otherwise minute devices. Additionally, certain changes in the shape of basket-shaped catheters due to contact with tissue are not efficiently detected with force sensors. Attempts to determine contact with tissue proximity indicators also have difficulties with dielectric variations in different types of tissue. The disclosure provides mechanisms and systems that can be applied to determine catheter contact or force with tissue and to determine change of shape catheter.

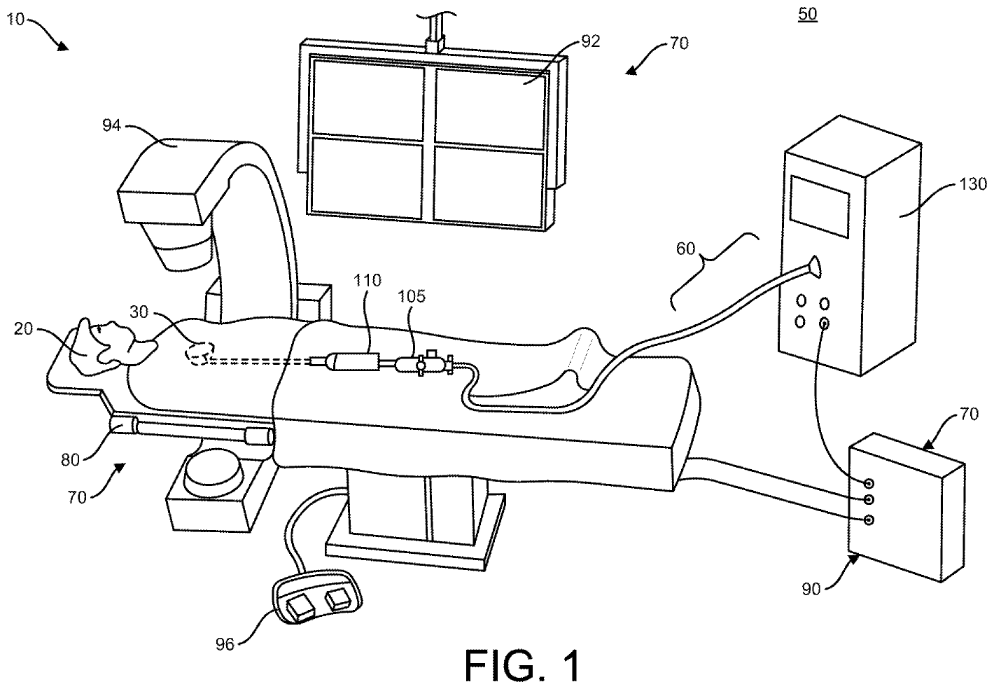

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver ablation electric field energy to targeted tissue in the patient's heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient's heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. In one example, the electroporation catheter 105 is a mapping and ablation catheter, which can be deployed in mapping procedures in cooperation with the EAM system 70 as well as to deliver ablation electric field energy and ablate tissue via irreversible electroporation.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end region configured to be deployed proximate target tissue, such as within a chamber of the patient's heart. The shaft can extend from an access point in the patient to the target tissue and generally defines a longitudinal axis of the electroporation catheter 105. The distal end region may include a basket, balloon, spline, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of splines configured to form a basket, and at least a some of the electrodes are disposed on the splines.

The electroporation catheter 105 is configurable in a plurality of positions. For example, when the distal end region of the catheter 105 is within the introducer sheath 110, such as to travel to the patient to the chamber of the heart, the electrode deployment mechanism and electrode assembly are in a collapsed position to fit within the introducer sheath 110. Once the catheter has reached the destination in the chamber of the heart, for example, the introducer sheath 110 is retracted from the distal end region of the catheter 105 (or the shaft catheter is extended past the introducer sheath 110) and the electrode deployment mechanism and electrode assembly can be configured in an expanded position. The electrode assembly has a collapsed shape when the catheter 105 is in the collapsed position and an expanded shape when the catheter 105 is in the expanded position. In some examples, the electrode assembly has more than two positions.

Additionally, the electrode deployment mechanism in this disclosure can be deformable such that the electrode deployment mechanism and electrode assembly can be in a deformed configuration that deviates from the expanded position. The electrode deployment mechanism is deformable with respect to the expanded position. For example, the electrode deployment mechanism and electrode assembly in the expanded position can become deformed when subjected to a force greater than a threshold force. In one instance, the electrode deployment mechanism and electrode assembly can be deformed if pressed or urged against target tissue such as a heart wall. For example, splines can be constructed from a deflectable or malleable material or constructed to include yieldable components. In certain examples, the electrode deployment mechanism is resilient and returns to the expanded position when the force is removed such as if the electrode deployment mechanism is moved away from the target tissue.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect electroporation or mapping.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

The electroporation console 130 can also receive electrical signals from the electrodes in the electric fields, such as electrical signals generated from the electric fields or in response to the electric fields. The electrical signals can be indicative of parameters of the electric fields. The electrical signals can be applied for feedback on electric field. Measurement or processing of the electrical signals can determine parameters of the electric fields such as locations of electrodes and field strength. For example, the electrodes can include ablation electrodes that are configured to deliver ablation electric field energy and mapping electrodes for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map, also referred to as an anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest.

The EAM system 70 is configured to generate the anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an anatomical map of the patient and, in some instances, on an electro-anatomical map of the patient's heart. The integrated system can include the capability to enhance the efficiency of clinical workflows, including enhancement of providing a visual representation to the clinician of ablation lesions of portions of the patient's heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

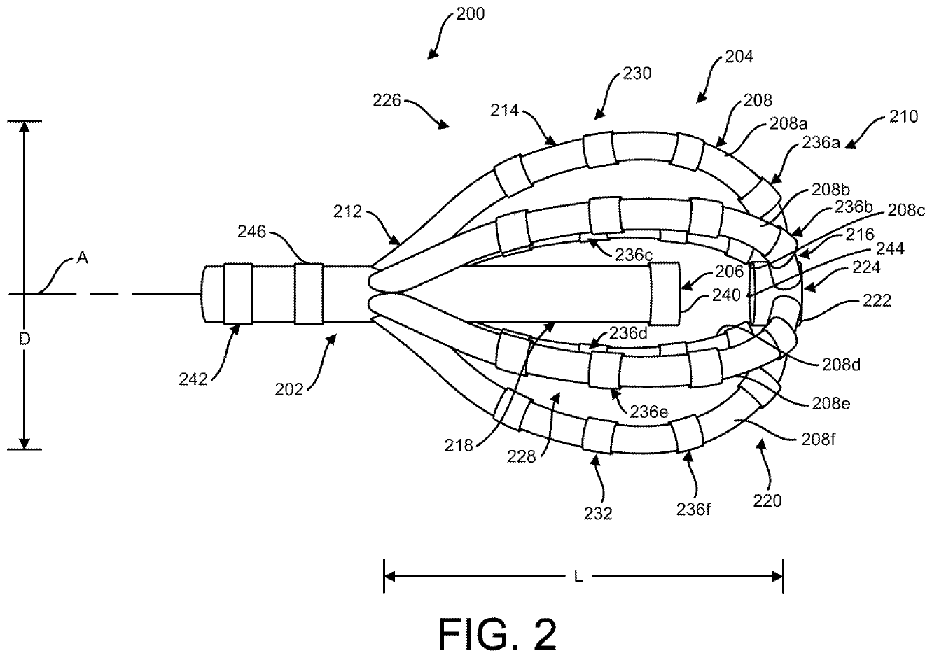

FIG. 2 illustrates an example electroporation catheter 200, which can be an example of catheter 105 and used with the electrophysiology system 50. The example electroporation catheter 200 includes an elongated catheter shaft 202 and distal end region 204 configured to be deployed proximate target tissue, such as within a chamber of the patient's heart. The shaft 202 can extend from an access point in the patient to the target tissue and generally defines a longitudinal axis A of the electroporation catheter 200. The longitudinal axis A is presented as a line passing through a centroid of a cross section of the shaft 202. The shaft 202 is terminated distally at a shaft end 206. The distal end region 204 in the example includes a plurality of splines 208, as illustrated splines 208a-208f, forming an electrode deployment mechanism such as a basket 210 in the example. The number of splines in the plurality of splines 208 is presented for illustration, and a basket 210 can also include more or fewer than the six illustrated spines. Each spline of the plurality of splines 208 includes a spline proximal end 212, a spline longitudinal portion 214 having a longitudinally extending length, and a spline distal end 216. In the example, the spline proximal end 212 of each of the plurality of splines 208 is attached to the shaft 202 proximal to the shaft end 206. In such an example, the shaft 202 includes a distal basket region 218 disposed within the basket 210 and termination within the basket 210. The spine distal end 216 of each of the plurality of splines are coupled together at a distal tip region 220 to form a basket distal end 222 and, in the example, a distal tip 224 of the catheter 200.

The catheter 200 is illustrated in an expanded position 226 in which the basket 210 is deployed for a procedure. As illustrated, the spline longitudinal portions 214 are spaced-apart from each other and the distal basket region 218 of the shaft 202 in the expanded position 226. The basket 210 in the expanded position can include an expanded length L and an expanded diameter D, and a particular contour. The length of the basket 210, such as length L, can be determined from the distance along the longitudinal axis A between the most-proximal proximal end 212 of splines 208 to the most-distal distal end 216 of the spines 208. The diameter of the basket 210, such as diameter D, can be determined from the greatest distance between longitudinal portions 214 of opposite spline 208 along a line generally perpendicular to the axis A. In the expanded position 226, the plurality of splines 208 are spaced-apart from each other and form a cavity 228 within the basket 210, and the distal basket region 218 of the shaft 202 is disposed within the cavity 228.

In the example, the plurality of spline 208 are constructed from a flexible and resilient material such that the catheter 200 can also be transitionable to a collapsed position (not shown) in which the collapse length of the basket, in this example, is greater than L and the collapsed diameter of the basket is less than D. In the collapsed position, the splines 208 may contact each other and the distal basket region 218 of the shaft 201. In one example, the catheter 200 is placed in the collapsed position while an introducer sheath, such as sheath 106, is disposed over the splines 208, and the catheter is in a non-operational mode. If the basket 210 is extended from the introducer sheath, or the introducer sheath is retracted from the basket 210, the basket can assume the expanded position 226 and be deployed in an operational mode.

In some examples, the catheter 200 includes a proximal end (not shown), and the catheter 200 may include a handle assembly in the proximal region of the catheter 200 coupled to the shaft 202 to allow a clinician to manipulate the catheter 200 during an electroporation ablation procedure. Additionally, the catheter can include a navigation sensor (not show) on or near the distal end region 204, which navigation sensor may be disposed within the shaft 202 such as within the distal basket region 218, The navigation sensor may collect sensor data and can be electrically coupled to the proximal end of the catheter via leads to provide electrical signals to the EAM system 70 regarding the location of the navigation sensor in the electrophysiology system 50.

The catheter 200 includes an electrode assembly 230 having a plurality of spaced-apart electrodes 232. The plurality of electrodes 232 can be formed of a conductive, solid-surface, biocompatible material. In the illustrated example, at least some of the plurality of electrodes 232 are ring electrodes disposed around the shaft 204 and splines 208, but other configurations are contemplated, and are spaced-apart across insulators the shaft 202 and splines 208. For instance, the shaft 202 and splines 208 may be covered with an electrically insulating material. Each of the plurality of electrodes 232 is electrically coupled to a corresponding elongated lead conductor disposed within shaft 204 that extend along the shaft 202 to the catheter proximal end. In one example, each electrode of the spaced-apart electrodes 232 corresponds with a separate, single lead conductor. In another example, a plurality of electrodes 232 may be coupled to a single lead conductor. Other configurations are contemplated. The plurality of lead conductors can be insulated from one another within an insulating sheath along the shaft 202, such as with an insulating polymer sheath. The lead conductors can be electrically coupled to plug in the proximal region of the catheter 200, such as a plug configured to be mechanically and electrically coupled to the electroporation console 130, for example, either directly or via intermediary electrical conductors such as cabling. In one example, the electroporation console 130 is configured to provide an electrical signal, such as a plurality of concurrent or space-apart-time electrical signals, to the electrically connected electroporation catheter 200 along lead conductors to the spaced-apart electrodes 232. The spaced-apart electrodes 232 are configured to generate a selected electrical field proximate the target tissue, based on the electrical signals from the electroporation console 130, such as to effect electroporation or mapping.

The electrode assembly 230 includes a plurality of spline electrodes 234 spaced-apart from each other in the expanded position and disposed on the spline longitudinal portions 214 of at least some of the plurality of splines 208. In the illustrated example, each spline 208a-208f includes a set of spaced-apart spline electrodes 236. For example, spline 208a includes a set of spline electrodes 236a. Spline 208b includes a set of spline electrodes 236b. Spline 208c includes a set of spline electrodes 236c. Spline 208d includes a set of spline electrodes 236d. Spline 208e includes a set of spline electrodes 236e. Spline 208f includes a set of spline electrodes 236f. The number of spline electrodes in each set of spline electrodes 236a-236f can vary and be other than four as illustrated. Further, the spacing between the spline electrodes along the longitudinal portions 214 can vary and be other than generally equidistant as illustrated. In one example, the spaced-apart set of spline electrodes 236 are fixed to the respective spline 208 so that each spine electrode in each set of spline electrodes is spaced-apart from every electrode in the same set of spline electrodes and no spline electrode in the set of spline electrodes 236 touches another spline electrode in the same set of spline electrodes. Further, each spline electrode in the plurality of spline electrodes 234 is spaced-apart from every spline electrode in the expanded state and no spline electrode of the plurality of spline electrodes touches another spline electrode in the expanded state. In a non-operational mode of the catheter 200, however, such as collapsed position, it is contemplated that electrodes may touch each other.

The electrode assembly 230 can include additional electrodes including, in some examples, a measurement indifferent electrode 240, or measurement electrode 240, and a shaft electrode 242. The measurement electrode 240 is disposed within the basket 210, such as in cavity 228 when the catheter 200 is in the expanded position, to be protected from touching target tissue while the catheter 200 is deployed in an operation mode within the heart. In the example, the measurement electrode 240 is included on the distal basket region 218 of the shaft 204 such that the measurement electrode is in the cavity 228 when the catheter 200 is in the expanded position. As illustrated, the example measurement electrode 240 is disposed on the shaft end 206 as a cap that includes a ring portion around a longitudinal side of the shaft end 206 and a major surface on the distal tip of the shaft end. Additionally, or in some examples alternatively to the measurement electrode 240, the electrode assembly 230 can include a distal indifferent electrode 244, or distal electrode 244, that can be coupled to the distal tip region 220 such as the basket distal end 222. In the example, the distal electrode 244 is coupled to the basket distal end 222 to be within the basket 210 such as in cavity 228 when the catheter 200 is in the expanded position. In the illustrated example, the distal electrode 244 includes a proximal-facing major surface that, when used in combination with the measurement electrode 240, faces the major surface of the measurement electrode 240. A spacing between the measurement electrode 240 and distal electrode 244 along axis A can be distance d. Additionally, or in some examples alternatively to the measurement electrode 240, the electrode assembly 230 can include the shaft electrode 242 disposed on the shaft 202 in a region proximal to the basket 210. The example catheter 200 includes a plurality of shaft electrodes, such as a pair of shaft electrodes 242, 246. The shaft electrodes 242, 246 in the example are ring electrodes. The example illustrates the measurement electrode 240 and the shaft electrodes 242, 246 affixed to the shaft 202, and thus in a fixed spatial relationship in the operating mode of the catheter 200. The basket 210, and the plurality of splines 208, are deflectable with relation to each other and the shaft 202, and thus the plurality of spline electrodes 234 and the distal electrode 244 are moveable with respect to each other and with respect to the measurement electrode 240 and shaft electrodes 242, 246.

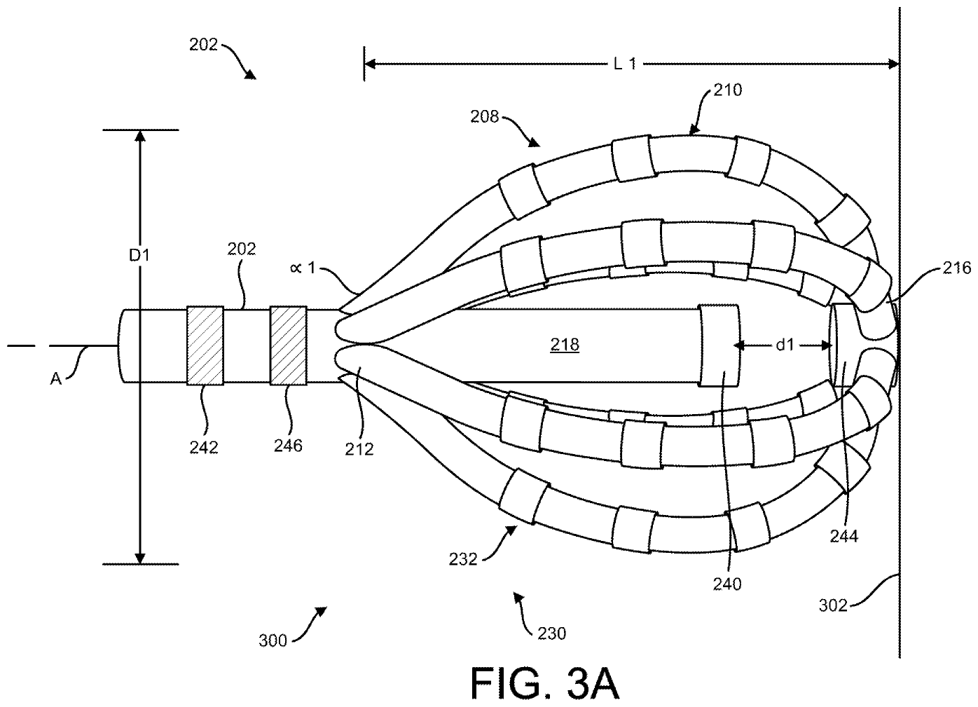

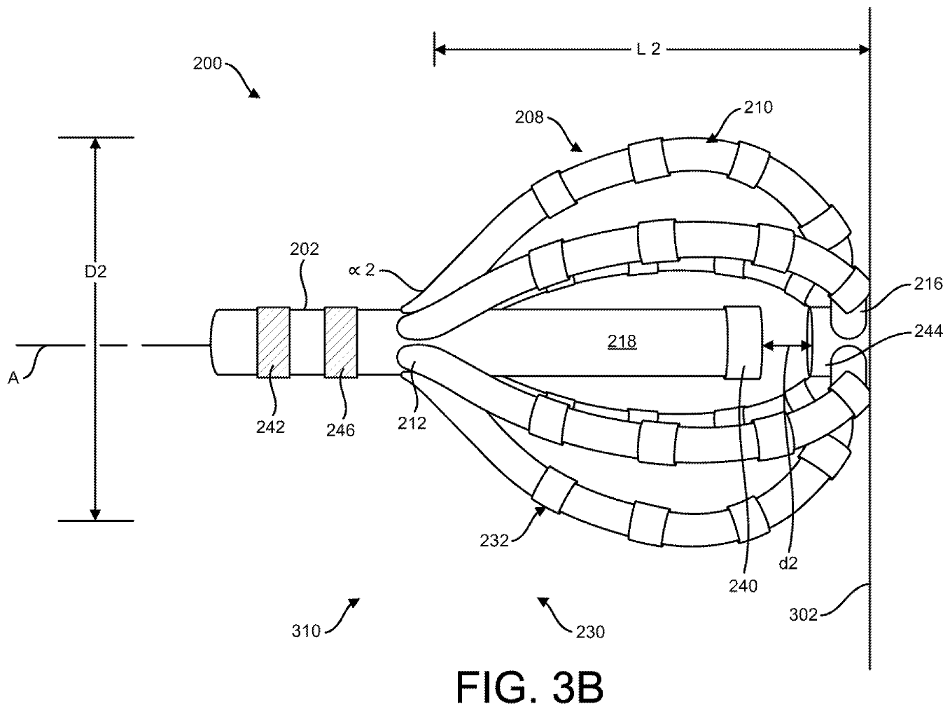

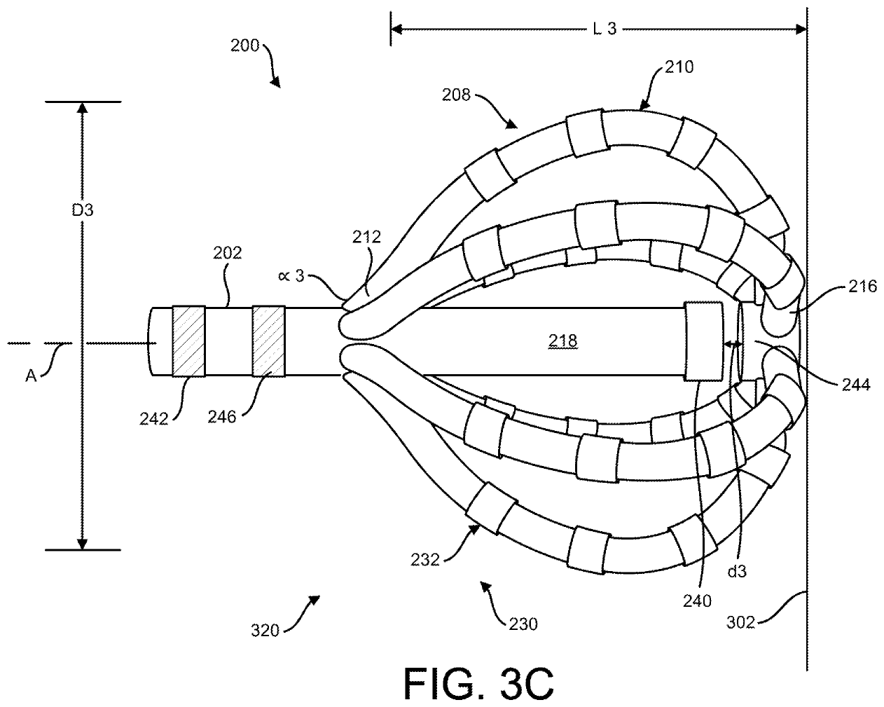

FIGS. 3A-3C illustrate the catheter 200 in various operational modes while deployed within an organ, such as a chamber of a patient's heart, in which like parts include like reference numerals. FIG. 3A illustrates the catheter 200 in an expanded position 300, which is a first operational mode, adjacent to a heart wall 302. In the expanded position 300, the basket 210 is either not subjected to a force or the force subjected to the basket 210 does not exceed a threshold force to cause a bending or deformation of a spline of the plurality of splines 208. For example, the catheter 200 can assume the expanded position 300 if the basket 210 is in the chamber away from the heart wall 302, or barely touching the heart wall 302. In the expanded position 300 the basket 210 includes a diameter of D1 and a length of L1, which correspond with diameter D and length L of FIG. 2. In the example, measurement electrode 240 and the distal electrode 244 are spaced apart by a distance d1. For the purposes of illustration, the splines 208 are shaped in the form of a Bezier curve—a parametric curve with a series of control points that defines a generally smooth, continuous arc—extending from the proximal end 212 to the distal end 216, wherein a characteristic of the Bezier curve is an alpha α1 at the proximal end 212. By way of illustration, example reference dimensions include a length L1 of 15 mm, diameter D1 of 10 mm, and distance d1 of 3 mm, and an alpha α1 of 0.80.

FIG. 3B illustrates the catheter 200 in a first deformed state 310, which is also an operational mode in that the electrodes 232 of the electrode assembly 230 are spaced apart from each other, adjacent to the heart wall 302. In the first deformed state 310, the basket 210 is adjacent to the heart wall 302 and is subjected to a force along the longitudinal axis A in the direction toward the distal tip 224 that exceeds the threshold force to cause a bending or deformation of a spline of the plurality of splines 208 from the expanded position 300. For example, the catheter 200 can assume the first deformed state 310 as the clinician begins to push the basket against the heart wall 302. In the first deformed state 310, the basket 210 includes a diameter of D2, which is greater than diameter D1 of the expanded position 300, and a length of L2, which is less than L1 of the expanded position 300. In the example, measurement electrode 240 and the distal electrode 244 are spaced apart by a distance d2, which is less than d1 of expanded position 300. A characteristic of the Bezier curve of the splines 208 in the first deformed state is alpha α2 at the proximal end 212, in which alpha α2 is greater than alpha α1 of the expanded position 300. By way of illustration, example reference dimensions include a length L2 of 14 mm, diameter D2 of 11 mm, and distance d2 of 2 mm, and an alpha α2 of 0.85. Here, certain electrodes of the plurality of electrodes 232, such as electrodes opposite each other on a spline 208, are closer together or closer to the measurement electrode 240 than in the expanded position 300. Also, certain electrodes of the plurality of electrodes 232, such as electrodes on different splines 208a-208f, are further apart than in the expanded position 300. The positions of at least some of the electrodes 208 in the electrode array of the first deformed state 310 are different in space than in the expanded position 300.

FIG. 3C illustrates the catheter 200 in a second deformed state 320, which is also an operational mode in that the electrodes 232 of the electrode assembly 230 are spaced apart from each other, adjacent to the heart wall 302. In the second deformed state 320, the basket 210 is also adjacent to the heart wall 302 and is subjected to a force along the longitudinal axis A in the direction toward the distal tip 224 that exceeds the threshold force to cause further bending or deformation of a spline of the plurality of splines 208 from the first deformed state 310. For example, the catheter 200 can assume the second deformed state 310 as the clinician continues to push the basket against the heart wall 302 after making strong contact with a force strong enough to bend the splines 208. In the second deformed state 320, the basket 210 includes a diameter of D3, which is greater than diameter D2 of the first deformed state 310, and a length of L3, which is less than L2 of the first deformed state 310. In the example, measurement electrode 240 and the distal electrode 244 are spaced apart by a distance d3, which is less than d2 of first deformed state 310. A characteristic of the Bezier curve of the splines 208 in the second deformed state 320 is alpha α3 at the proximal end 212, in which alpha α3 is greater than alpha α2 of the first deformed state 310. By way of illustration, example reference dimensions include a length L3 of 13 mm, diameter D3 of 12 mm, and distance d3 of 1 mm, and an alpha α3 of 0.90. Here, certain electrodes of the plurality of electrodes 232, such as electrodes opposite each other on a spline 208, are closer together or closer to the measurement electrode 240 than in the first deformed state 310. Also, certain electrodes of the plurality of electrodes 232, such as electrodes on different splines 208a-208f, are further apart than in the first deformed state 310. The positions of at least some of the electrodes 208 in the electrode array of the second deformed state 320 are different in space than in the expanded position 300 or the first deformed state 310.

An electrical field is generated in the electrode assembly 220, and signals received from electrodes 232 in the electrical field can be measured and used to determine or ascertain the positions of the electrodes 232 of the electrode assembly 230 in space. For example, the electrical field can be measured to determine parameters of the electric filed, and the parameters of the electric field can be used to determine or ascertain the positions of the electrodes 232 of the electrode assembly 230 in space with respect to a reference point on the catheter 200 such as the measurement electrode 240, shaft electrode 242, or other reference point. In one example, a first group of electrodes of the electrode assembly 230 can be used to measure an electric field generated by the electrode assembly 230, and ascertained positions of the electrodes can used to determine whether the basket 210 is in the expanded position 300, or in a deformed state, such as states 310, 320, relative to the expanded position 300. In one example, the first group of electrodes can include the measurement electrode 240, the shaft electrodes 242, 246, the measurement electrode 240 and shaft electrodes 242, 246, or another electrode in the electrode assembly. A determination that the basket 210 is in a deformed state can be used as an inferential determination that the basket 210 is in contact with a heart wall or other boundary, and a determination that the basket 210 is in an expanded position can be used as an inferential determination that the basket is not in contact with tissue or barely in contact with tissue. Based on such inferential determinations, along with navigational information from the catheter 200, the electroanatomical map can be updated with indications that the catheter 200 is at a boundary in the location of the basket 210.

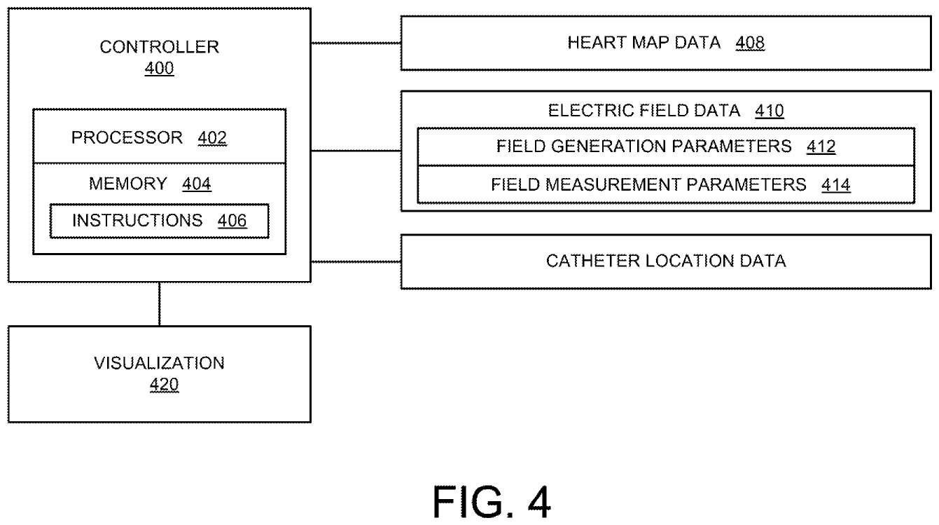

FIG. 4 illustrates a functional block diagram of an example controller 400 that can be used with the example electrophysiology system 50, such as a controller of the example electroporation catheter system 60 or a controller of the example EAM system 70, which can include a controller of the electroporation console 130 or the mapping and navigation controller 90, respectively. In other examples, controller 400 can be a controller of an integrated electroporation catheter system 60 and EAM system 70 or a controller for use with the electroporation catheter system 60 and EAM system 70.

The controller 400 can be implemented to indicate that the catheter 105 is in contact with the heart, such as with a wall of a chamber of the heart, as well as to indicate a degree or an amount of the contact based on deformation of the catheter. In some examples, the controller 400 can also be implemented to indicate where within the heart the catheter is in contact with tissue. In some examples, the controller can be implemented to indicate an amount of tissue distension or compression after contact with the catheter has been established. The amount of deformation of the catheter can be used to generate more accurate anatomical maps of the heart or to amend anatomical maps of the heart. Deformation of the catheter 105 can be an inferential measurement of force or contact with the tissue.

The controller 400 can include a processor 402 and a memory 404. The memory 404 stores processor executable instructions 406. In one example, the processor executable instructions can be in the form of a program, such as a computer program or application. The processor 402 can execute the instructions 406 that can be included in configuring the controller 400. In one example, the controller 400 can be implemented to include a computing device such as a laptop computer, a workstation, a desktop computer, a tablet, or a smartphone. In such examples, the controller 400 can include additional components such as a display, a touchscreen, speakers or other output devices, a keyboard or other input devices, or communication circuitry such as computer network adapters. The controller 400 may be implemented in a variety of architectures and components, such as the processor 402 and memory 404, may be distributed in various locations.

In one example, the processor 402 may include a plurality of main processing cores to run an operating system and perform general-purpose tasks on an integrated circuit. The processor 402 may also include built-in logic or a programmable functional unit, also on the same integrated circuit with a heterogeneous instruction-set architecture. In additional to multiple general-purpose, main processing cores and the application processing unit, controller 400 can include other devices or circuits such as graphics processing units or neural network processing units, which may include heterogeneous or homogenous instruction set architectures with the main processing cores. For example, the controller 400 may be used to perform other tasks such as in the case of a computing device including the resonance sound amplification device.

Memory 404 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 402. Any such computer storage media may be part of the controller 300 and implemented as memory 404. Memory 404 is a non-transitory, processor readable memory device. Accordingly, a propagating signal by itself does not qualify as storage media or memory 304.

Processor-executable instructions 406 may include, for example, computer code or machine-useable instructions such as, for example, program components capable of being executed by the processor 402 associated with controller 400. Program components may be programmed using any number of different programming environments, including various languages, development kits, or frameworks. Some or all of the program components can also, or alternatively, be implemented in hardware.

The controller 400 may be configured to receive inputs or information from the electrophysiology system 50, such as inputs from the electroporation catheter system 60 and EAM system 70 including the electroporation console 130 and the mapping and navigation controller 90, for storage in memory 404 and use by the instructions 406. For example, the controller 400 can receive an input representative of the anatomical map of the heart, or heart map data 408, which heart map data 408 can include the data regarding representations of the geometric anatomical map of the heart and the electro-anatomical map of the heart, such as from the EAM system 70. Heart map data 408 can be stored in memory 404. Additionally, the controller can receive electric field parameter data 410, such as from the electroporation catheter system 60, and the electric field parameter data 410 can be stored in memory 404. Electric field parameter data 410 can include electric field generation parameter data 412 and includes electric field measurement parameter data 414. Inputs and information can include user generated information to add to or annotate data 408, 410, 412.

Electric field generation parameter data 412 can include data regarding parameters of the electric field as provided to be produced or generated by the catheter 105 in the patient's heart. In this example, the electric field to be generated by the catheter is of a strength less than used for irreversible electroporation. In one example, the controller 400 can cause the electroporation catheter system 60 to generate the electric field. For instance, the controller 400 determines the parameters of the electric field and provides signals regarding the electric field generation parameter data 412 to the electroporation catheter system 60 to generate the electric field in the catheter 105 in response to the electric field generation parameter data 412. In another example, the electroporation catheter system 60 generates the electric field and provides the electric field generation parameter data 412 to the controller 400. Electric field generation parameter data 412 can include such parameters as data regarding the strength of the electric field intended to be generated by the catheter 105, the strength of the signals provided to the electrode assembly, the configuration of the electrodes in the electrode assembly, such as whether an electrode in the electrode assembly is configured as an anode or a cathode. In other example, the electric field generation parameter data 412 can include information regarding the catheter 105, such as the configuration and spacing of the electrodes on the catheter 105. Examples of configuration and spacing of the electrodes on the catheter 105 can include information regarding the spacing and sizing of the electrodes in the electrode assembly in the expanded position, the size of the electrodes, and whether an electrode is applied as a measurement electrode 240 or a shaft electrode 242, 246 such as on catheter 200, the nominal spacing of the electrodes on the splines in the expanded position and the spacing of the splines with respect to each other in the expanded position, and the distance between electrodes such as distance between the measurement electrode 240 and the distal electrode in the expanded position, and the distance between measurement electrode 240 and any shaft electrodes 242, 246 on the catheter shaft 202.

Electric field measurement parameter data 414 includes data regarding parameters of the electric field as measured or determined by the catheter 105 in the patient's heart in response the generated electric field. Electric field measurement parameter data 414 can include such parameters as data regarding the strength of the electric field as measured by the catheter 105, the strength of the signals provided to the electrode assembly such as the electrical parameters of signals received from individual electrodes of the electrode assembly, the configuration of the electrodes in the electrode assembly, such as whether an electrode in the electrode assembly is configured as an anode or a cathode or a measurement electrode or a shaft electrode such as on catheter 200, for example. The electric field measurement parameter data 414 can include other data from the electric field as measured by the catheter 105 in the patient's heart. For instance, the measurement electrode 240, the distal electrode 244, and shaft electrodes 242, 246 when immersed in an electric field can be used to provide signals that can be interpreted as electric field measurement parameter data 414. In one example, the electrical signal measured corresponds with a voltage. The measurement electrode 240 is disposed within the basket 210 so as not to touch heart tissue, which can be beneficial in providing a reference point as well as providing electric field measurement parameter data 414. In other examples, a spline electrode 236 or set of spline electrodes can be designated to provide signals that can be interpreted as electric field measurement parameter data 414.

The controller 400 can receive an input representative of the relative location of the catheter 105, such as a location within the patient's heart, or within tracking system, as catheter location data 416. In one example, a location sensor or sensing element on the electroporation catheter 105, can generate an output that can be processed by the controller 400 to track the location of the sensor, and consequently, the catheter 105, within the localization volume. The location sensors or sensing elements on the catheter 105 can include magnetic devices or electrodes, for example.

Based on electric field generation parameter data 412, includes electric field measurement parameter data 414, and catheter location data 416, the controller can determine information such as the location of electrodes 232 with respect to each other and the location of the catheter. In one example, the electric field generation parameter data 412, includes electric field measurement parameter data 414, and catheter location data 416, the controller can determine information such as the location of measurement electrodes, such as measurement electrode 240, which does not contact tissue within the heart chamber. Additionally, the location of the distal electrode 244 with respect to the measurement electrode 240 can be determined. Further, the respective locations of the electrodes on a spline, such as the locations of a set of spline electrodes 236 with respect to each other. Still further, the respective locations of a group of spline electrodes can be determined with respect to other groups of spline electrodes, such as group of spline electrodes 236a with respect to other groups of spline electrode 236b-236f. With enough measurement signals from the electrodes to provide electric field measurement parameter data 414, the location shape of the basket 210 can be determined and the amount of force used to deform the basket, from below a threshold force to a force to place the catheter in a non-operative state, can be determined.

The controller 400 is configured to generate a visualization 420 that can include information related to how much the catheter is deformed from the expanded position. In one example, the controller 400 is configured to generate in the visualization 420 the amount of deformation, such as with a deformation gradient on a display. In another example, the controller 400 is configured to generate in the visualization 420 a real-time rendering of the deformation in the catheter, such as highlighting portions of the catheter that have been deformed with respect to the expanded position. In still another example, the controller 400 is configured to generate in the visualization 420, in connection with the heart map data 408, an update in the heart map on a graphical display and store such information into memory 404.

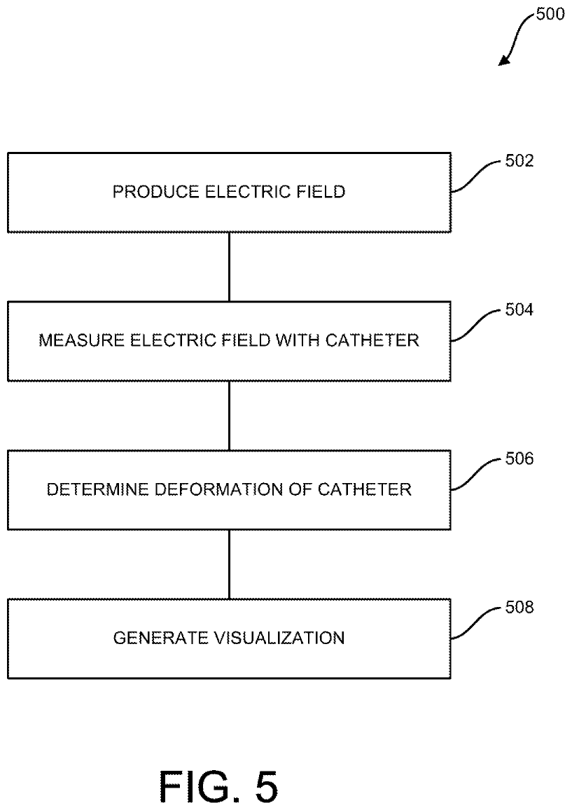

FIG. 5 illustrates a process 500 of configuring a controller, such as controller 400, while performing an electroporation of target tissue, such as in a chamber of a patient's heart. In one example, the controller is implemented as part of the EAM system 70 and operably coupled to the electroporation catheter system 60. Process 500 includes configuring the controller to produce an electric field in a catheter at 502. For instance, the catheter can be an example of catheter 105, such as catheter 200, and can include an electrode assembly 230 having a plurality of spaced-apart electrodes 232 disposed on a plurality of splines 208 to generate an electric field in which the splines 208 are configurable in an expanded position 300 such that the plurality of spaced-apart electrodes are in a selected spatial relationship. The splines 208 are deformable relative to the expanded position 300 when subjected to a force and can assume deformed states, such as deformed states 310, 320. In one example, the catheter includes a shaft 200 having a distal region 204, and the plurality of splines 208 form a basket 210 in the expanded position 300, wherein each of the plurality of splines 208 includes a proximal end 212 and a distal end 216, and the basket 210 is coupled to the distal region 204 wherein the distal ends 216 of the splines 208 form a distal tip region 222 of the basket 210. The controller 400 is configured to receive and process information regarding the generated electric field with electric field generation parameter data 412.

The controller 400 is configured to measure an electrical signal received from an electrode of the plurality of spaced-apart electrodes 232 in response to the electric field, in which the electrical signal indicative of a parameter of the electric field at 504. In one example of 504, the controller 400 is configured to measure a plurality of electrical signals received from the plurality of spaced-apart electrodes 232 in response to the electric field, the plurality of electrical signals indicative of parameters of the electric field. In one example, the plurality of electrodes includes a measurement electrode 240 disposed within the basket 210 and configured to not contact tissue, such as heart wall 302, when the catheter 200 is in the expanded position 300 that can be used to measure the electric signal. In one example, the plurality of electrodes includes a distal indifferent electrode, such as distal electrode 244, disposed within the basket 210 and coupled to the distal tip region 222 of the basket 210, that can be used to measure an electric signal. Additionally, the plurality of electrodes 232 includes a shaft electrode 242 or 246 disposed on the distal region of the shaft and proximal to the basket that can be used to measure an electric signal. For example, the controller is configured to receive and process the measured electrical signals as electric field measurement parameter data 414.

The deformation of the splines is determined based on a determination, from the measured electrical signal, of the locations of the plurality of electrodes. In one example, a change in the measured electrical signals over time is used to determine deformation. In another example, a determination can be made as to whether the basket is in the expanded position or in a deformed but operational state based on the measured signals. For instance, the deformation of the splines is determined from a distance between the plurality of spaced-apart electrodes based on the measured electrical signal. From the determined deformation, a determination can be made that the basket is in contact with a structure, such as the heart wall. In one example, the determination of the deformation includes a determination of an amount of deformation of the splines relative to the expanded position, such as a degree of deformation. From the degree of deformation, a determination can be made as to the amount of force being applied to the heart wall with the basket.

The controller 400 is configured to determine a deformation of the splines relative to the expanded position based on the measured electrical signal at 506. In one example, the controller can apply electric field generation parameter data 412 regarding the catheter, such as the configuration and spacing of the electrodes on the catheter in the expanded position to determine whether the catheter is in a deformed state. Examples of configuration and spacing of the electrodes on the catheter 200 can include information regarding the spacing and sizing of the electrodes in the electrode assembly in the expanded position 300, the size of the electrodes, and whether an electrode is applied as a measurement electrode 240 or a shaft electrode 242, 246 such as on catheter 200, the nominal spacing of the electrodes on the splines in the expanded position and the spacing of the splines with respect to each other in the expanded position, and the distance between electrodes such as distance between the measurement electrode 240 and the distal electrode 244 in the expanded position, and the distance between measurement electrode 240 and any shaft electrodes 242, 246 on the catheter shaft 202.

In one example, the controller 400 is configured to determine a deformation at 506 via selecting a reference electrode from the plurality of electrodes 232. In one example, the reference electrode can be any one or more electrodes of the plurality of electrodes.

For instance, the reference electrode can be the measurement electrode 240, which can provide a robust measurement because it is disposed within the basket and configured to not contact tissue when the catheter is in the expanded position and in many deformed states. From measured electrical signals, such as electric field measurement parameter data 414, the controller is configured to determine the relative spatial location of each electrode in the plurality of electrodes with respect to the reference electrode. The controller can continually determine the relative spatial locations of the electrodes and determine whether there are changes to the relative spatial locations. The controller can cycle through selected pairs of electrodes to determine deformation. A deformation to the basket can be determined if a change to a relative spatial location surpasses a selected threshold amount. In one example, the measurement electrode 240 and the distal electrode 244 are used as reference electrodes. Additionally, a determination that the measurement electrode 240 and distal electrode 244 are in contact with each other can be used to determine deformation. In still another example, the shaft electrodes can be used as a reference electrode in a separate or second determination of deformation.

In one example of a determination of deformation at 506, the controller is configured to apply the measured electrical signal to a look up table having a set of signals corresponding with a deformed state or amount of deformation. For instance, the lookup table can be populated with nodes of signals corresponding to the deformation state and amount of deformation or force applied from a testing stage of the system. During an ablation procedure, the measured electrical signal is input into the lookup table and a corresponding output is provided. Measured electrical signals not matching with a node may be converted to a determination of deformation based on a nearby node with adjustments to the corresponding amount of deformation. In one example, the system can apply multiple lookup tables and access a corresponding lookup table based on other parameters of the system as determined from electric field generation parameter data 412, for example, such as a particular lookup table for a variety of catheter, a particular lookup table for electric field strength applied in mapping, a particular lookup table for the configuration of electrodes used to perform the map, and other lookup tables.

In one example, the determination of the deformation includes a determination of distension of tissue such as after contact with tissue or deformation of the splines is determined. Electrode signals from measured signals at 504, catheter location data 416, and catheter shape information from the determination of deformation at 506 can be applied to determine distension of tissue. As tissue is distended, the tissue will contact more electrodes 232 independent of the basket compression or decrease in the distance d between the measurement electrode 240 and the distal electrode 244. An amount of tissue distension can be estimated based on an amount of changes to electrodes in contact with tissue and changes in catheter location adjusted for basket shape deformation.

The controller 400 is configured to generate a visualization based on the deformation at 508. The visualization can be on a graphical display and can be in addition to or an overlay on the electroanatomical map and use catheter location data 416 to determine the location of the catheter. In some examples, the visualization can include a real-time changes. For example, the controller is configured to generate a visualization of a gradient based on the amount of deformation of the splines relative to the expanded position. The gradient can be a bar graph on a graphical display. The controller can also be configured to generate a visualization of the deformation of the splines on the graphical display. The visualization can highlight a deformed spline of the plurality of splines in the visualization of the deformation of the splines. The visualization of the deformation of the splines can also be rendered on the electroanatomical map such as with heart map data 408 and catheter location data 416. In one example, the controller is configured to determine the deformation of the splines relative to the expanded position in substantially real time.

In one example, process 500 can be implemented as set of processor-executable instructions, such as instructions 406, stored in a non-transitory memory, such as memory 404 to be executed by a processor 402 to configure controller 400. The instructions to implement process 500 can be configured to receive information, such as to retrieve from memory 404, heart map data 408, electric field parameter data 410 including electric field measurement parameter data 414, and catheter location data 416. Further, the instructions to implement process 500 can be configured to annotate, adjust, or write to heart map data 408 and to generate a visualization, such as visualization 420 on a display of graphical representations.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system for facilitating an ablation, the system comprising:
a catheter including an electrode assembly having a plurality of spaced-apart electrodes disposed on a plurality of splines to generate an electric field, the splines configurable in an expanded position wherein the plurality of spaced-apart electrodes are in a selected spatial relationship, and wherein the splines are deformable relative to the expanded position when subjected to a force; and
a controller operably coupled to the electrode assembly, the controller configured to:
measure an electrical signal received from an electrode of the plurality of spaced-apart electrodes in response to the electric field, the electrical signal indicative of a parameter of the electric field; and
determine a deformation of the splines relative to the expanded position based on the measured electrical signal.

2. The system of claim 1, wherein the catheter includes a shaft having a distal region, and the plurality of splines form a basket in the expanded position, wherein each of the plurality of splines includes a proximal end and a distal end, the basket coupled to the distal region wherein the distal ends of the splines form a distal tip region of the basket.

3. The system of claim 2, wherein the plurality of electrodes includes a measurement electrode disposed within the basket and configured to not contact tissue when the catheter is in the expanded position.

4. The system of claim 3, wherein the measurement electrode provides the electrical signal.

5. The system of claim 3, wherein the plurality of electrodes includes a distal indifferent electrode disposed within the basket and coupled to the distal tip region, and the controller is configured to determine a distance between the measurement electrode and the distal indifferent electrode.

6. The system of claim 1, wherein the deformation of the splines is determined based on a determination, from the measured electrical signal, of a location of each of the plurality of electrodes.

7. The system of claim 1, wherein the determination of the deformation includes a determination of an amount of deformation of the splines relative to the expanded position.

8. The system of claim 7, wherein the controller is configured to determine an amount of the force applied to the splines based on the determination of an amount of deformation.

9. The system of claim 1, wherein the controller is configured to generate a visualization based on the deformation.

10. The system of claim 9, wherein the controller is configured to generate a visualization of a gradient based on the amount of deformation of the splines relative to the expanded position.

11. The system of claim 9, wherein the controller is configured to highlight a deformed spline of the plurality of splines.

12. The system of claim 9, wherein the controller is configured to generate the visualization of the deformation on an electroanatomical map of a heart.

13. An electroporation catheter for use with tissue, comprising:
an elongated shaft having a distal region; and
an electrode assembly operably coupled to the distal region, the electrode assembly having a plurality of spaced-apart electrodes disposed on a plurality of splines to generate an electric field, the splines configurable in an expanded position wherein the plurality of spaced-apart electrodes are in a selected spatial relationship, and wherein the splines are deformable relative to the expanded position when subjected to a force;
wherein the plurality of splines form a basket defining a cavity in the expanded position, wherein each of the plurality of splines includes a proximal end and a distal end, the basket coupled to the distal region wherein the distal ends of the splines form a distal tip region of the basket, the shaft having a distal basket region extending into and terminating within the cavity; and
wherein the plurality of electrodes includes a measurement electrode disposed within the basket on the distal basket region of the shaft, the measurement electrode configured to not contact the tissue when the splines are in the expanded position.

14. The catheter of claim 13, wherein the plurality of electrodes includes a distal indifferent electrode disposed within the basket and coupled to the distal tip region, the distal indifferent electrode spaced-apart from the shaft and the measurement electrode.

15. The catheter of claim 13, wherein the plurality of electrodes includes a shaft electrode disposed on the distal region of the shaft and proximal to the basket.

16. A process for facilitating an ablation in a patient's heart, the process comprising:
generating an electric field with a catheter in the heart, the catheter including an electrode assembly having a plurality of spaced-apart electrodes disposed on a plurality of splines to generate the electric field, the splines configurable in an expanded position wherein the plurality of spaced-apart electrodes are in a selected spatial relationship, and wherein the splines are deformable relative to the expanded position when subjected to a force; and
measuring an electrical signal received from an electrode of the plurality of spaced-apart electrodes in response to the electric field, the electrical signal indicative of a parameter of the electric field;
determining a deformation of the splines relative to the expanded position based on the measured electrical signal wherein the deformation of the splines is determined based on determining, from the measured electrical signal, a location of each of the plurality of electrodes with respect to the electrode assembly.

17. The process of claim 16, and further including determining an amount of the force applied to the catheter based on the determination of an amount of deformation.

18. The process of claim 16, and further including generating, on a graphical display, a visualization based on the deformation.

19. The process of claim 18, wherein generating the visualization includes generating the visualization of the deformation of the splines on an electroanatomical map of a heart.

20. The process of claim 16, wherein determining deformation further includes determining an amount of tissue distension based on a determined amount of the plurality of electrodes in contact with the heart, a determined location of the catheter within the heart, and a determined amount of deformation.
